(19) United States
(12) Patent Application Publication
(10) Pub. No.: US 2021/0386921 A1
(43) Pub. Date: Dec. 16, 2021

(54) SYSTEM FOR PERITONEAL DIALYSIS

(71) Applicant: KAISEN TECHNOLOGY SDN BHD, Petaling Jaya, Selangor (MY)

(72) Inventor: Khing Hong LOKE, Petaling Jaya (MY)

(73) Assignee: KAISEN TECHNOLOGY SDN BHD, Petaling Jaya, Selangor (MY)

(21) Appl. No.: 17/290,202

(22) PCT Filed: Oct. 30, 2019
(86) PCT No.: PCT/MY2019/000043

§ 371 Date: Apr. 29, 2021

(30) Foreign Application Priority Data
Oct. 30, 2018 (MY) PI 2018704019

(51) Int. Cl. A61M 1/28

(52) U.S. Cl. CPC A61M 1/281; A61M 2205/3331; A61M 2205/3553; A61M 2230/50; A61M 2205/8206; A61M 2230/201; A61M 2230/04; A61M 2205/583

(57) ABSTRACT

The present invention relates to a system for peritoneal dialysis, more particularly the present invention relates to a system for delivering dialysis fluid or solution to a patient comprising a device (100) for receiving dialysis fluid from a fluid package containing the dialysis fluid and delivering the dialysis fluid to a patient via a delivery channel and draining out toxins and excess fluid from the patient via a draining channel into a draining package.

### FIELD OF INVENTION

The present invention relates to a system for peritoneal dialysis, more particularly the present invention relates to a system for delivering dialysis fluid or solution to a patient.

### BACKGROUND ART

Dialysis is procedure for removing toxins from the body. More particularly, the peritoneal dialysis (PD) is a procedure for removing toxins from the blood that takes advantage of the semi-permeable membrane surrounding the walls of the abdomen or peritoneal cavity.

During a PD procedure, a solution is introduced into the patients abdomen, where it remains in the body for up to several hours, removing blood toxins via osmotic transfer through the membrane. At completion of the procedure, the solution is drained out from the body along with the toxins. In an automated PD, the procedure is handled by an automated equipment.

There are systems in the market for performing automated PD. Such systems include diaphragm pumps and a variety of other complex components. The cost is an ever-increasing issue, and PD equipment manufacturers have attempted to reduce production costs by several means. One is to employ peristaltic pumps, though, they have inherent limitations. For example, the approach of accurately measuring the fluid volume delivered by a peristaltic pump can be difficult, because the tubing in the pump loses elasticity and, consequently, its volume changes over time. The PD pumps also tend to develop pinhole leaks in pump tubing.

There is therefore a need for a system for delivering dialysis fluids to a patient in an automated peritoneal dialysis procedure that addresses the above-mentioned complications.

### SUMMARY OF INVENTION

The present invention aims to provide a system for delivering dialysis fluid or solution to a patient, in an effective, safe and comfortable manner. In particular, the present invention discloses a system for an improved automated peritoneal dialysis.

It is an object of the present invention to provide a system for peritoneal dialysis comprising a device for receiving dialysis fluid from a fluid package containing the dialysis fluid and thereafter delivering the dialysis fluid to a patient via a delivery channel and draining out toxins and excess fluid from the patient via a draining channel into a draining package for disposal.

It is another object of the present invention to provide a system for peritoneal dialysis comprising a device for receiving dialysis fluid from a fluid package containing the dialysis fluid and thereafter delivering the dialysis fluid to a patient via a delivery channel and draining out toxins and excess fluid from the patient via a draining channel into a draining package for disposal, wherein the device further comprises a fill sensor for monitoring pressure of the delivery channel in the event of delivering the dialysis fluid, and a drain sensor for monitoring pressure of the draining channel in the event of draining out the toxins and excess fluid, and the device is configured for delivering the dialysis fluid to the patient in a manner that the delivery is based on clinical conditions of the patient.

The present invention addresses the solution of providing a system which is configured to overcome the complications of the existing automated peritoneal dialysis systems.

### BRIEF DESCRIPTION OF THE ACCOMPANYING DRAWINGS

To further clarify various aspects of some embodiments of the present invention, a more particular description of the invention will be rendered by references to illustrations in the appended drawings. It is appreciated that these drawings depict only typical embodiments of the invention and are therefore not to be considered limiting of its scope. The invention will be described and explained with additional specificity and detail through the accompanying drawings in which:

FIG. 1 illustrates a visual representation of the device in accordance with the preferred embodiment of the present invention.

FIG. 2 illustrates a schematic representation of the auxiliary devices being connected to a smartphone in accordance with the preferred embodiment of the present invention.

FIG. 3 illustrates a s schematic representation of a real-time cloud-based information sharing system for remote patient monitoring and prescription in accordance with the preferred embodiment of the present invention.

FIG. 4 illustrates a schematic representation of the system layout in accordance with the preferred embodiment of the present invention.

### DETAILED DESCRIPTION OF THE INVENTION

Reference will now be made in detail to the preferred embodiments of the present invention, and results of which are illustrated in the accompanying drawings.

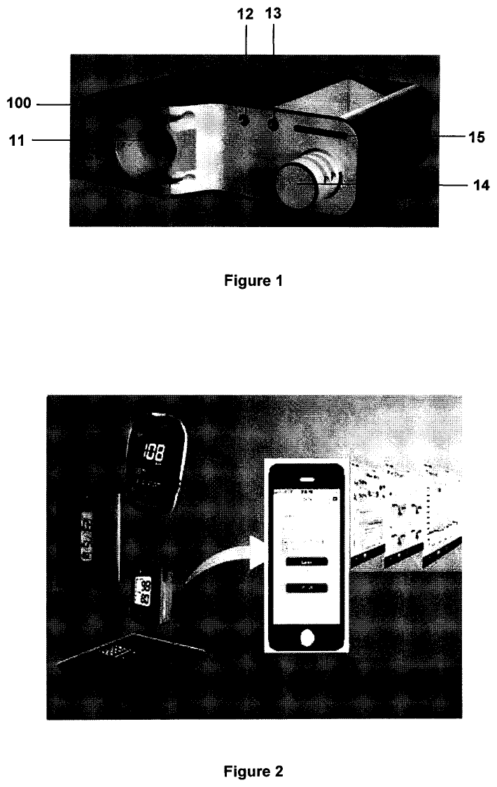

Referring to FIG. 1, the figure illustrates a visual representation of the device (100) of a system for peritoneal dialysis according to the preferred embodiment of the present invention.

The device (100) is also known as an Ultra-portable Automated Peritoneal Dialysis Cycler. The device (100) of the present invention is for receiving dialysis fluid or dialysis solution, from a fluid package or a solution bag, containing the dialysis fluid and thereafter delivering the dialysis fluid to a patient via a delivery channel and draining out toxins and excess fluid from the patient via a draining channel into a draining package or a draining bag.

The device (100) in the preferred embodiment of the present invention, further comprises a fill sensor (12) for monitoring pressure of the delivery channel in the event of delivering the dialysis fluid, and a drain sensor (13) for monitoring pressure of the draining channel in the event of draining out the toxins and excess fluid, and the device (100) is configured for delivering the dialysis fluid to the patient in a manner that the delivery is based on clinical conditions of the patient, such that the delivery of dialysis fluid is specific to a patient's clinical conditions.

The sensors, mainly the fill sensor (12) or known as the filling pressure sensor and control system, and the drain sensor (13) or known as the peritoneum pressure sensor and control sensor, are the dual sensors in the preferred embodiment of the present invention to provide primary safety feature for the patient's safety and comfort. The filling pressure sensor and control system is to monitor the pressure in the delivery channel during the filling process of the dialysis fluid for patient safety and to prevent situation of over filling and vice versa for over draining via the draining channel. The peritoneum pressure and control sensor will assist in the complete draining of the dialysis fluid for maximum ultrafiltration effect.

The device (100) preferably comprises a dual energy (AC/DC) power and low ampere management system, of which the device (100) is preferably powered by any one of an alternating current (AC) power source, a direct current (DC) power source, or a power bank device. The dual energy power management system provides high portability of the device (100), and such the peritoneal dialysis procedure can be performed in remote location with no national grid power supply. The capability of the device (100) of being powered by low amperes (less than 2.7 A) allows the device (100) to be powered by 20,000 mAh of electric current (24V output voltage) via a small rechargeable power bank for up to 3 to 4 days of dialysis procedures. The maximum power consumption of the device (100) is about 65 Watts.

Further, the device (100) in the preferred embodiment of the present invention, comprises a pump mechanism (11) or known as a dual directions positive-pressure pump system, which is configured for pumping fluid in dual direction positive pressure. The pump mechanism (11) comprises a main positive pressure pump system that delivers the dialysis fluid from the fluid package to the peritoneum via the delivery channel and discharge drain fluids from peritoneum to the draining package via the draining channel. The delivery channel and the draining channel are configured within a Y-connector tubing system with an infection detection kit. The pump mechanism (11) is capable of regulating preferably but no limited to 3 modes of speed, that is fast, medium and slow during both filling and draining processes. The 3 modes of speed offer flexible timing for completion of the full procedure of dialysis ranging from 6, 7 and 8 hours, unlike in the prior art which is fixed at a standard of 8 to 10 hours. The pump mechanism (11) is capable of introducing multiple steps of acceleration speed and deceleration speed for both the filling and draining processes for the patient's safety and comfort. In the event of a malfunction of the pump mechanism (11) and is unable to rotate the motor, the pump mechanism (11) in the preferred embodiment of the present invention is configured to stop the filling and draining processes so as to and eliminate risks of over filling or over draining.

The Y-connector tubing system with an infection detection kit are provided to deliver the dialysis fluid to the patient and drain the toxins and excess fluid from the patient, with a safety feature incorporated. The Y-connector tubing system with an infection detection kit, eliminate the use of cartridges and cassettes of the prior art. The infection detection kit is capable of testing and determining infection in peritoneum via the drain fluids for early infection detection. Also, the device (100) of the preferred embodiment of the present invention comprises a flow control mechanism (14) configured for controlling flow of fluid in dual direction. The flow control mechanism (14) is configured with feedback sensors for opening and closing the delivery channel and the draining channel to provide the required delivery of the dialysis fluid and draining of the toxin. The flow control mechanism (14) is the main traffic control system to regulate the flowing of the dual directions of the dialysis fluid and the draining of toxins during the filling and draining processes. The device (100) further comprises a magnetic calibration and validation mechanism, which are the secondary safety feature, wherein the magnetic calibration and validation mechanism is for determining rotation of a pump head and a peristaltic motor, and accuracy of the rotation as predetermined by a software program.

Referring to FIG. 2, the figure illustrates a schematic representation of the auxiliary devices being connected to a smartphone in accordance with the preferred embodiment of the present invention. In a further configuration of the device (100), the device (100) is connectable to auxiliary devices via wired connection or wireless connection. The device (100) is preferably configured for connection with a plurality of wireless devices, which are either or a combination of Bluetooth and other Internet of Things (IoT) enabled devices, smartphones or tablets.

A first device of the plurality of wireless devices is preferably but not limited to a device with a body weighing scale, a thermometer, a blood pressure meter, a blood glucose meter, a blood cholesterol or an acid uric meter. A second device of the plurality of wireless devices is preferably but not limited to a NB-IoT (Narrowband IoT) device for location tracking and identification even within enclosed building by detecting surrounding internet protocol (IP) address for location identification. A third device of the plurality of wireless devices is preferably but not limited to an image capturing device which is integrated within smartphones and tablets that is capable of capturing image of the result of the infection detection kit and the information will be sent via mobile applications and cloud computing so as to allow real-time remote monitoring. A fourth device of the plurality of wireless devices is preferably but not limited to smartphones or tablets with near field communication (NFC) reader, Bluetooth or radio frequency identification (RFID) systems, and the device is preferably configured with a verification module for verifying the dosage and expiry date of the dialysis fluid prior to commencement of the dialysis procedure for reducing medical error. The verification module provides confirmation of information of the patient, dosage, and procedure duration for the peritoneal dialysis. Further, the device (100) is configured with a RFID sensor for tracking information of the RFID label on the peritoneal dialysis solution bags and sharing the information with the cloud-based real-time remote patient management system.

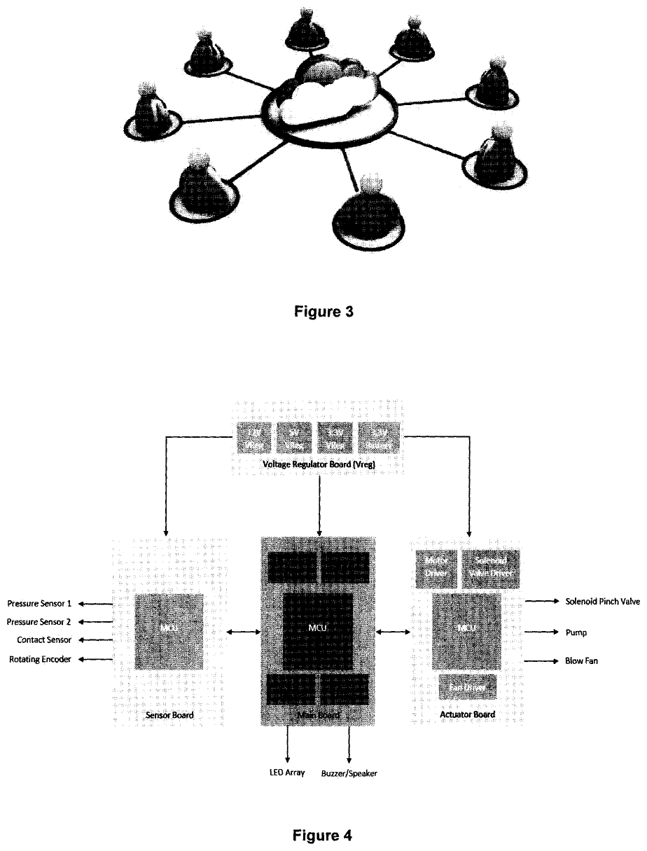

Referring to FIG. 3, the FIG. 3 illustrates a s schematic representation of a real-time cloud-based information sharing system for remote patient monitoring and prescription in accordance with the preferred embodiment of the present invention

In the preferred embodiment of the present invention, the device (100) is preferably operated via applications of smartphones or tablets for real-time operation and for remotely prescribing dialysis delivery, whereby the applications are used to electronically provide or modify prescriptions by a nephrologist in a telehealth environment using the telemedicine application. The applications provide two-way artificial intelligence communication system that support autonomous collection of patient personal health information such as fluctuation of body weight, body temperature, blood pressure, blood glucose level, acid uric and cholesterol level for cloud storage which will subsequently allow real-time remote patient monitoring by a dedicated renal nurse or a dedicated physician.

Moreover, the preferred system of the present invention is customizable to provide varying personalized profile of the dialysis procedure for a better clinical outcome. The system is capable of customizing flexible filling amount of the dialysis fluid, such as 7 liters, 8 liters, 9 liters or 10 liters as prescribed using the telemedicine application, and not to the standard 10 liters in the prior art. The reduction of amount as prescribed by the physician and together with the new packing size of the dialysis fluid will result in lesser wastage, and will contribute towards better utilization of water and energy resources. Also, the system is capable of customizing flexible duration for the dialysis procedure, such as 6 to 8 hours according to a user's preference, and not the standard 8 to 10 hours of procedure in the prior art. The flexible duration of procedure, especially shorter hours will offer better quality of life to the patients.

There are 3 standard profile for the peritoneal dialysis procedure in the prior art as follows:

i) Full Fill Full Drain Profile

| | | | Ultrafiltration |
|---|---|---|---|
| | Fill Volume | Drain Volume | (extra fluid removed) |
| Cycler | (Liters) | (Liters) | by calculated formula |
| 1 | 2 | 2 | By estimate** |
| 2 | 2 | 2 | By estimate** |
| 3 | 2 | 2 | By estimate** |
| 4 | 2 | 2 | By estimate** |
| 5 | 2 | 2 | By estimate** |
| Total | 10 | 10 | By estimate** |

The prior art is incapable of achieving draining of the fluid until empty.

ii) Keep the Last Fill Profile

| | | | Ultrafiltration |
|---|---|---|---|
| | Fill Volume | Drain Volume | (extra fluid removed) |
| Cycler | (Liters) | (Liters) | by calculated formula |
| 1 | 2 | 2 | By estimate** |
| 2 | 2 | 2 | By estimate** |
| 3 | 2 | 2 | By estimate** |
| 4 | 2 | 2 | By estimate** |
| 5 | 2 | *** | By estimate** |
| Total | 10 | 8 | By estimate** |

The balance 2 liters of the drain volume will be manually drained out by the patient without using the device of the prior art.

iii) Tidal Dialysis

| | | | Ultrafiltration |
|---|---|---|---|
| | Fill Volume | Drain Volume | (extra fluid removed) |
| Cycler | (Liters) | (Liters) | by calculated formula |
| 1 | 2 | 1.5 | Balance in peritoneum |
| 2 | 1.5 | 1.5 | Balance in peritoneum |
| 3 | 1.5 | 1.5 | Balance in peritoneum |
| 4 | 1.5 | 1.5 | Balance in peritoneum |
| 5 | 1.5 | 1.5 | Balance in peritoneum |
| 6 | 1.5 | 1.5 | Balance in peritoneum |
| 7 | 0.5 | 1 | Balance in peritoneum |
| Total | 10 | 10 | |

The prior art is incapable of draining the fluid to empty, and thus the tidal dialysis will maintain a small volume of fluid in the peritoneum until the last cycle. Some balance dialysis fluid will be kept in the peritoneum and only remove during the last cycle.

As can be seen in the prior art procedure profiles, the dialysis procedures are not customizable as described in the preferred embodiment of the present invention. The system of the present invention is customizable and provides personalized operation of the peritoneal dialysis procedure, in which pressure sensors are configured to monitor and regulate the intraperitoneal pressure ranging from 2.12 kPa (8.5″ H2O or 0.31 psi) to 7.93 kPa (31.83″ H2O or 1.15 psi) pressure during filling of peritoneal dialysis solution into peritoneum of the patient or draining drain fluid or ultrafiltration from the peritoneum of the patient. The system of the present invention improves the number of peritoneal dialysis exchange cycles for the patient by reducing the overall draining and filling time phase in each of the cycle.

The device (100) in the preferred embodiment of the present invention also comprises at least a visual indicator (15) for indicating status of the device (100) to a user. The device (100) is configured with preferably but not limited to Bluetooth and status indicators with the use of multiple colours of a single LED indicator to indicate the status of completion or readiness of the device (100) for performing step-by-step functions.

The device (100) which intelligently synchronizes the operation of the dialysis procedures, weighs preferably less than 2.4 kg and has a dimension of less than 210 mm×297 mm×120 mm, which makes the device (100) highly portable and automated.

Referring to FIG. 4, the figure illustrates a schematic representation of the system layout in accordance with the preferred embodiment of the present invention, wherein the system comprises a voltage regulator module, controllers, sensors and components for the automated operation of the preferred system of the present invention.

The present invention may be embodied in other specific forms without departing from its essential characteristics. The described embodiments are to be considered in all respects only as illustrative and not restrictive. The scope of the invention is, therefore indicated by the appended claims rather than by the foregoing description. All changes, which come within the meaning and range of equivalency of the claims, are to be embraced within their scope.

1. A system for peritoneal dialysis comprising:
a device (100) for receiving dialysis fluid from a fluid package containing the dialysis fluid and delivering the dialysis fluid to a patient via a delivery channel and draining out at least a toxin from the patient via a draining channel into a draining package;
characterized in that the device (100) further comprises a fill sensor (12) for monitoring pressure of the delivery channel in the event of delivering the dialysis fluid, and a drain sensor (13) for monitoring pressure of the draining channel in the event of draining out said toxin, and the device (100) is configured for delivering the dialysis fluid to the patient in a manner that the delivery is based on clinical conditions of the patient.

2. The system according to claim 1, wherein said device (100) is powered by any one of an alternating current power source, a direct current power source, or a power bank device.

3. The system according to claim 1, wherein said device (100) further comprises a pump mechanism (11) configured for pumping fluid in dual direction positive pressure.

4. The system according to claim 1, wherein the delivery channel and the draining channel are configured within a Y-connector tubing system with an infection detection kit.

5. The system according to claim 1, wherein said device (100) further comprises a flow control mechanism (14) configured for controlling flow of fluid in dual direction.

6. The system according to claim 1, wherein the device (100) further comprises a magnetic calibration and validation mechanism for determining rotation of a pump head and a peristaltic motor, and accuracy of the rotation as predetermined.

7. The system according to claim 1, wherein the device (100) is connectable to auxiliary devices via wired connection or wireless connection, and said auxiliary devices further comprise at least one device configured with a verification module.

8. The system according to claim 1, wherein the device (100) is configured for real-time operation for remotely prescribing dialysis delivery and for remotely monitoring the patient.

9. The system according to claim 1, wherein the device (100) further comprises pressure sensors configured to monitor and regulate the intraperitoneal pressure during filling of peritoneal dialysis solution into peritoneum of the patient or draining drain fluid or ultrafiltration from the peritoneum of the patient.

10. The system according to claim 1, wherein the device (100) is portable and automated.
